(19) United States
(12) Patent Application Publication
(10) Pub. No.: US 2008/0228069 A1
(43) Pub. Date: Sep. 18, 2008

(54) Novel Differential Imaging Method

(75) Inventors: Jagat Narula, Santa Ana, CA (US); Ignasi Carrio, Barcelona (ES); Arnold Jacobson, Yardley, PA (US)

Correspondence Address: GE HEALTHCARE, INC., IP DEPARTMENT, 101 CARNEGIE CENTER, PRINCETON, NJ, 08540-6231, US

(73) Assignee: MEDIPHYSICS, INC., Princeton, NJ (US)

(21) Appl. No.: 10/580,941

(22) PCT Filed: Nov. 29, 2004
(86) PCT No.: PCT/US2004/039832

§ 371 Date: May 28, 2008

Related U.S. Application Data
(60) Provisional application No. 60/526,019, filed on Dec. 1, 2003.

(51) Int. Cl. A61B 5/055

(52) U.S. Cl. 600/431; 382/131; 324/309

(57) ABSTRACT

The present invention relates to an improved method of imaging cardiac neurotransmission in vivo in a human subject using adrenergic imaging agents. The method comprises obtaining two separate images with the same adrenergic imaging agent. One of the images is obtained in conjunction with the administration of a compound known to interfere with the uptake of the particular imaging agent in question. Comparison of the two images enables additional information to be obtained in relation to the status of cardiac neurotransmission in said subject compared with imaging with adrenergic imaging agent alone. The invention also provides a method of imaging cardiac neurotransmission in a human subject in vivo wherein a single image is obtained using an adrenergic imaging agent in conjunction with the administration of a non-pharmaceutical dose of an agent known to interfere with the uptake of the imaging agent. The invention furthermore provides a method of operating an imaging apparatus, a second medical use of an adrenergic imaging agent as well as a kit suitable for carrying out the methods of the invention.

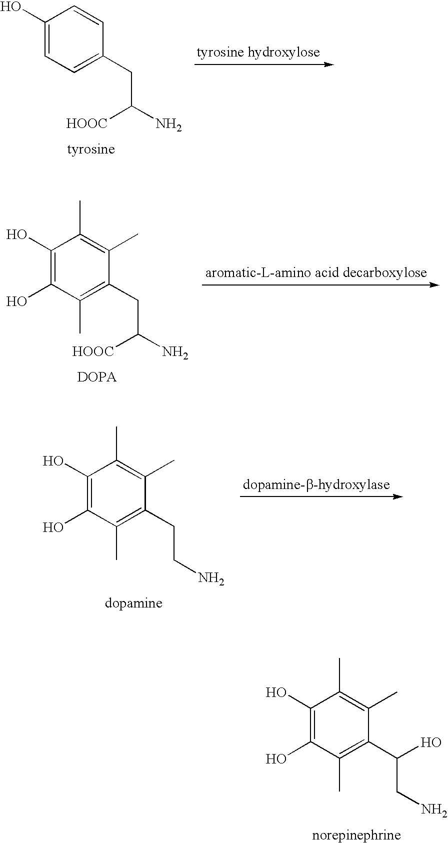

### TECHNICAL FIELD OF THE INVENTION

The present invention relates to the field of medical imaging of human subjects. In particular the invention relates to cardiac neurotransmission imaging in said subjects and provides a novel imaging method that provides further clinical data compared with known imaging methods.

### DESCRIPTION OF RELATED ART

Various radiopharmaceuticals are known that target the tissues involved in cardiac neurotransmission, and are therefore useful in the diagnosis and monitoring of diseases where this function is compromised. Examples of such radiopharmaceuticals are ¹⁸F-fluorodopamine, ¹¹C-hydroxyephidrine (¹¹C-HED), ¹¹C-ephidrine (¹¹C-EPI), ¹²³I-meta-iodobenzylguanidine (¹²³I-mIBG), ¹¹C-4-(3-t-butylamino-2-hydroxypropoxy)-benzimidazol-1 (¹¹C-CGP), ¹¹C-carazolol, ¹⁸F-fluorocarazolol and ¹¹C-methylquinuclidinyl benzylate (¹¹C-MQNB). Use of these radiopharmaceuticals permits the in vivo assessment of presynaptic reuptake and neurotransmitter storage in addition to the regional distribution and activity of postsynaptic receptors. Radiopharmaceuticals labelled with ¹²³I can be used for external imaging using single photon emission computed tomography (SPECT) and those labelled with ¹¹C or ¹⁸F can be used for external imaging using positron emission tomography (PET). For a recent review of the characteristics and uses of these agents see Carrió, Journal of Nuclear Medicine 2001 42(7) pp 1062-76.

Many classes of medicines are known to interfere with the uptake of the above mentioned radiopharmaceuticals, e.g. tricyclic antidepressants, beta blockers, calcium channel blockers, sympathomimetic agents and cocaine. The discontinuation of these potentially interfering medicines prior to the administration of one of said radiopharmaceuticals has been strongly advised in order to decrease the likelihood of a false negative result (Solanki et al, Nuclear Medicine Communications 1992 13 pp 513-21, Kurtaran et al European Journal of Radiology, 2002 41 pp 123-30, CIS-US Inc. “Iobenguane Sulfate ¹³¹I Injection Diagnostic” pack insert July 1999).

### SUMMARY OF THE INVENTION

The present invention relates to an improved method of imaging cardiac neurotransmission in vivo in a human subject using imaging agents. The method comprises obtaining two separate images with the same imaging agent. One of the images is obtained in conjunction with the administration of an agent known to interfere with the uptake of the particular imaging agent in question. Comparison of the two images enables additional information to be obtained in relation to the status of cardiac neurotransmission in said subject. In an intact neuron interference with uptake of the agent does not alter the uptake efficiency. In contrast, where there is a defect resulting in cardiac neurotransmission either working at maximal capacity at rest or rendered less efficient, uptake of the agent is significantly altered by the interfering agent. The invention also provides a method of imaging cardiac neurotransmission in a human subject in vivo wherein a single image is obtained using an imaging agent in conjunction with the administration of a non-pharmaceutical dose of an agent known to interfere with the uptake of the imaging agent. The invention furthermore provides a method of operating an imaging apparatus as well as a kit suitable for carrying out the methods of the invention.

### DETAILED DESCRIPTION OF THE INVENTION

In a first aspect the present invention relates to a method of assessing cardiac neurotransmission of a human subject comprising;
i) administration to said subject of an amount suitable for in vivo imaging of an adrenergic imaging agent;
ii) in vivo imaging of said subject using said adrenergic imaging agent;
iii) administration of an adrenergic interfering agent to said subject;
iv) repeating steps (i) and (ii); and,
v) comparing the images obtained in steps (ii) and (iv).

It is also envisaged that the method can be carried out where step (iii) is performed as the first step.

In the context of the present invention the term “cardiac neurotransmission” includes all those processes involved in the normal functioning of adrenergic neurons in the heart. Particular processes of interest in the context of the present invention are the synthesis, storage, release, reuptake and metabolism of norepinephrine (NE).

NE is synthesized from the amino acid tyrosine which is taken up by an active transport system into neurons from the blood stream (see FIG. 1 for synthetic route). Once inside the neuron, the aromatic ring of tyrosine is hydroxylated by the enzyme tyrosine hydroxylase to form dihydroxyphenylalanine (DOPA). DOPA is then acted upon by aromatic-L-amino acid decarboxylase to form dopamine (DA). DA is taken up into synaptic vesicles and converted to NE by β-hydroxylation mediated by dopamine-β-hydroxylase. NE is stored in the synaptic vesicles until required for use.

In healthy tissues, adrenergic neurons are stimulated to release NE from synaptic vesicles and into the synapse in response to certain stimuli such as exercise, fear and anxiety. The released NE acts to excite or inhibit organs depending on the receptors present on a particular cell type, i.e. α-1 and β-1 receptors produce excitation and α-2 and β-2 receptors cause inhibition.

Following its deployment to the synapse and receptors, NE is mainly taken back into the neurons by the energy-dependent sodium-dependent uptake-1 system. Once back in the neuron, NE is either taken up once more into the synaptic vesicles, or metabolized by monoamineoxidase (MAO) to form dihydroxyphenylglycol (DHPG), which is released into the bloodstream.

An extraneuronal uptake of NE can also occur, so-called “uptake-2”, which is energy-independent. This uptake mechanism becomes predominant at relatively high levels of NE. Once inside the non-neuronal cells, metabolism of NE takes place via the MAO pathway as well as via catechol-O-methyltransferase (COMT), which is responsible for the metabolism of NE to form lipophilic metabolite normetanephrine (NMN), which is released into the bloodstream. For a review of the biochemistry of NE see Eisenhofer et al (Review in Endocrine & Metabolic Disorders 2001 2 pp 297-311).

The term “adrenergic imaging agent” in the present invention is taken to mean an agent, labelled with an imaging moiety that can image adrenergic neurons. Typically such an agent interacts with a process of cardiac neurotransmission in a subject, and in particular processes relating to the synthesis, storage, release, reuptake and metabolism of NE, thereby enabling the assessment of cardiac neurotransmission in said subject. Suitable adrenergic imaging agents of the present invention include labelled forms of: neurotransmitter analogues, e.g. fluorodopamine (F-DOPA); false neurotransmitters, e.g. ephedrine (EPI), hydroxyephidrine (HED), meta-iodobenzylguanidine (mIBG) and meta-fluorobenzylguanidine (mFBG); agonists of β-adrenoreceptors, e.g. 4-(3-t-butylamino-2-hydroxypropoxy)-benzimidazol-1 (CGP), carazolol and fluorocarazolol; and muscarinic receptor antagonists, e.g. methylquinuclidinyl benzylate (MQNB). The term “labelled forms” in the context of the present invention is taken to mean forms labelled with an imaging moiety.

The “imaging moiety” enables detection following administration of said adrenergic imaging agent to the subject in vivo and is chosen from:
(i) a radioactive metal ion;
(ii) a paramagnetic metal ion;
(iii) a gamma-emitting radioactive halogen;
(iv) a positron-emitting radioactive non-metal;
(v) a hyperpolarized NMR-active nucleus;
(vi) a reporter suitable for in vivo optical imaging;
(vii) a β-emitter suitable for intravascular detection.

The imaging moiety may be detected either external to the human body or via use of detectors designed for use in vivo, such as intravascular radiation or optical detectors such as endoscopes, or radiation detectors designed for intra-operative use. Preferred imaging moieties are those which can be detected externally in a non-invasive manner following administration in vivo. Most preferred imaging moieties are radioactive, especially gamma-emitting radioactive halogens and positron-emitting radioactive non-metals, particularly those suitable for imaging using SPECT or PET.

When the imaging moiety is a radioactive metal ion, i.e. a radiometal, suitable radiometals can be either positron emitters such as ⁶⁴Cu, ⁴⁸V, ⁵²Fe, ⁵⁵Co, ^{94m}Tc or ⁶⁸Ga; γ-emitters such as ^{99m}Tc, ¹¹¹In, ^{113m}In, or ⁶⁷Ga. Preferred radiometals are ^{99m}Tc, ⁶⁴Cu, ⁶⁸Ga and ¹¹¹In. Most preferred radiometals are γ-emitters, especially ^{99m}Tc.

When the imaging moiety is a paramagnetic metal ion, suitable such metal ions include: Gd(III), Mn(II), Cu(II), Cr(III), Fe(III), Co(II), Er(II), Ni(II), Eu(III) or Dy(III). Preferred paramagnetic metal ions are Gd(III), Mn(II) and Fe(III), with Gd(III) being especially preferred.

When the imaging moiety is a gamma-emitting radioactive halogen, the radiohalogen is suitably chosen from ¹²³I, ¹³¹I or ⁷⁷Br. A preferred gamma-emitting radioactive halogen is ¹²³I.

When the imaging moiety is a positron-emitting radioactive non-metal, suitable such positron emitters include: ¹¹C, ¹³N, ¹⁵O, ¹⁷F, ¹⁸F, ⁷⁵Br, ⁷⁶Br or ¹²⁴I.

Preferred positron-emitting radioactive non-metals are ¹¹C, ¹³N and ¹⁸F, especially ¹¹C and ¹⁸F, most especially ¹⁸F.

When the imaging moiety is a hyperpolarized NMR-active nucleus, such NMR-active nuclei have a non-zero nuclear spin, and include ¹³C, ¹⁵N, ¹⁹F, ²⁹Si and ³¹P. Of these, ¹³C is preferred. By the term “hyperpolarized” is meant enhancement of the degree of polarization of the NMR-active nucleus over its' equilibrium polarization. The natural abundance of ¹³C (relative to ¹²C) is about 1%, and suitable ¹³C-labelled compounds are suitably enriched to an abundance of at least 5%, preferably at least 50%, most preferably at least 90% before being hyperpolarised.

When the imaging moiety is a reporter suitable for in vivo optical imaging, the reporter is any moiety capable of detection either directly or indirectly in an optical imaging procedure. The reporter might be a light scatterer (e.g. a coloured or uncoloured particle), a light absorber or a light emitter. More preferably the reporter is a dye such as a chromophore or a fluorescent compound. The dye can be any dye that interacts with light in the electromagnetic spectrum with wavelengths from the ultraviolet light to the near infrared. Most preferably the reporter has fluorescent properties.

Preferred organic chromophoric and fluorophoric reporters include groups having an extensive delocalized electron system, eg. cyanines, merocyanines, indocyanines, phthalocyanines, naphthalocyanines, triphenylmethines, porphyrins, pyrilium dyes, thiapyriliup dyes, squarylium dyes, croconium dyes, azulenium dyes, indoanilines, benzophenoxazinium dyes, benzothiaphenothiazinium dyes, anthraquinones, napthoquinones, indathrenes, phthaloylacridones, trisphenoquinones, azo dyes, intramolecular and intermolecular charge-transfer dyes and dye complexes, tropones, tetrazines, bis(dithiolene) complexes, bis(benzene-dithiolate) complexes, iodoaniline dyes, bis(S,O-dithiolene) complexes. Fluorescent proteins, such as green fluorescent protein (GFP) and modifications of GFP that have different absorption/emission properties are also useful. Complexes of certain rare earth metals (e.g., europium, samarium, terbium or dysprosium) are used in certain contexts, as are fluorescent nanocrystals (quantum dots).

Particular examples of chromophores which may be used include: fluorescein, sulforhodamine 101 (Texas Red), rhodamine B, rhodamine 6G, rhodamine 19, indocyanine green, Cy2, Cy3, Cy3.5, Cy5, Cy5.5, Cy7, Marina Blue, Pacific Blue, Oregon Green 488, Oregon Green 514, tetramethylrhodamine, and Alexa Fluor 350, Alexa Fluor 430, Alexa Fluor 532, Alexa Fluor 546, Alexa Fluor 555, Alexa Fluor 568, Alexa Fluor 594, Alexa Fluor 633, Alexa Fluor 647, Alexa Fluor 660, Alexa Fluor 680, Alexa Fluor 700, and Alexa Fluor 750.

Particularly preferred are dyes which have absorption maxima in the visible or near infrared region, between 400 nm and 3 μm, particularly between 600 and 1300 nm.

Optical imaging modalities and measurement techniques include, but not limited to: luminescence imaging; endoscopy; fluorescence endoscopy; optical coherence tomography; transmittance imaging; time resolved transmittance imaging; confocal imaging; nonlinear microscopy; photoacoustic imaging; acousto-optical imaging; spectroscopy; reflectance spectroscopy; interferometry; coherence interferometry; diffuse optical tomography and fluorescence mediated diffuse optical tomography (continuous wave, time domain and frequency domain systems), and measurement of light scattering, absorption, polarization, luminescence, fluorescence lifetime, quantum yield, and quenching.

When the imaging moiety is a β-emitter suitable for intravascular detection, suitable such β-emitters include the radiometals ⁶⁷Cu, ⁸⁹Sr, ⁹⁰Y,¹⁵³Sm, ¹⁸⁶Re, ¹⁸⁸Re or ¹⁹²Ir, and the non-metals ³²P, ³³P, ³⁸S, ³⁸Cl, ³⁹Cl, ⁸²Br and ⁸³Br.

Preferred imaging moieties of the present invention are those that can be detected external to the human body, with gamma-emitting radioactive halogens and positron-emitting radioactive non-metalsa being especially preferred.

When the imaging moiety is a radioactive halogen, such as iodine, a precursor of the adrenergic imaging agent is chosen to include: a non-radioactive halogen atom such as an aryl iodide or bromide (to permit radioiodine exchange); an activated aryl ring (e.g. a phenol group); an organometallic precursor compound (e.g. trialkyltin or trialkylsilyl); or an organic precursor such as triazenes. Methods of introducing radioactive halogens (including ¹²³I and ¹⁸F) are described by Bolton (2002 J Lab Comp Radiopharm. 45 pp 485-528). Examples of suitable aryl groups to which radioactive halogens, especially iodine can be attached are given below:

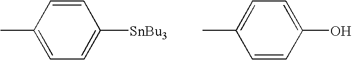

Both contain substituents which permit facile radioiodine substitution onto the aromatic ring. Alternative substituents containing radioactive iodine can be synthesized by direct iodination via radiohalogen exchange, e.g.

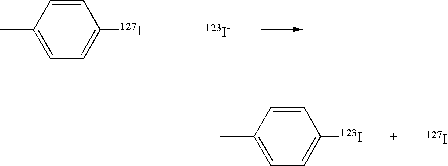

When the imaging moiety is a radioactive isotope of iodine the radioiodine atom is preferably attached via a direct covalent bond to an aromatic ring such as a benzene ring, or a vinyl group since it is known that iodine atoms bound to saturated aliphatic systems are prone to in vivo metabolism and hence loss of the radioiodine.

When the imaging moiety comprises a radioactive isotope of fluorine (e.g. ¹⁸F), the radioiodine atom may be carried out via direct labelling using the reaction of ¹⁸F-fluoride with a suitable precursor having a good leaving group, such as an alkyl bromide, alkyl mesylate or alkyl tosylate. ¹⁸F can also be introduced by N-alkylation of amine precursors with alkylating agents such as ¹⁸F(CH₂)₃OMs (where Ms is mesylate) to give N—(CH₂)₃¹⁸F, or O-alkylation of hydroxyl groups with ¹⁸F(CH₂)₃OMs or ¹⁸F(CH₂)₃Br. For aryl systems, ¹⁸F-fluoride displacement of nitrogen from an aryl diazonium salt is a good route to aryl-¹⁸F derivatives. See Bolton (2002 J. Lab. Comp. Radiopharm. 45 pp 485-528) for a description of routes to ¹⁸F-labelled derivatives.

Preferred adrenergic imaging agents of the present invention are ¹⁸F-fluorodopamine, ¹¹C-HED, ¹¹C-EPI, ¹²³I-mIBG, ¹³¹I-mIBG, ¹⁸F-mFBG, ¹⁸F-pFBG, ¹⁸F-FIBG, ¹¹C-CGP, ¹¹C-carazolol, ¹⁸F-fluorocarazolol and ¹¹C-MQNB. The most preferred agents of the present invention are ¹²³I-mIBG and ¹⁸F-mFBG, with ¹²³I-mIBG being especially preferred. Further detail in relation to these preferred imaging agents is provided in the following paragraphs and some of their structures are illustrated in FIG. 2.

Synthesis of ¹⁸F-fluorodopamine can be conveniently carried out by enzymatic decarboxylation of ¹⁸F-fluoro-DOPA using an L-amino acid decarboxylase (Luxen et al 1990 Int J Rad Appl Instrum. 41 pp 275-81), or alternatively by direct fluorination of dopamine (Chirakal et al Nuc Med Biol 1996 23 pp 41-5). ¹⁸F-fluorodopamine can be used in the assessment of NE synthesis as it participates in that process in the same way as dopamine. It is taken up in sympathetic nerve terminals and transported into synaptic vesicles where it is converted into ¹⁸F-fluoro-NE and stored. In a similar manner to NE, ¹⁸F-fluoro-NE is released from sympathetic nerve terminals upon sympathetic stimulation. ¹⁸F-fluorodopamine can be used in the evaluation of cardiac autonomic innervation in a variety of cardiac diseases with involvement of neuronal innervation.

¹¹C-EPI and ¹¹C-HED can be synthesized respectively by the methods outlined in Chakraborty et al (1993 Nucl Med Biol 20 pp 939-44) and Rosenspire et al (1990 J Nucl Med 31 pp 1328-34). ¹¹C-EPI and ¹¹C-HED can be used to assess the NE uptake and storage mechanisms as they are transported via uptake-1 into the neuron and in a similar manner to NE are stored in synaptic vesicles. ¹¹C-EPI, but not ¹¹C-HED, is metabolized by the same pathways as NE and therefore can act as a tracer for these pathways as well. ¹¹C-HED enables imaging of alterations in neuronal innervation in diabetes, congestive heart failure and after heart transplantation.

Radioiodinated mIBG can be synthesized according to the method described in Kline et al (1981 J Nucl Med. 22 pp 129-32). Methods of preparing carrier-free radioiodinated mIBG have also been reported, e.g. by Samnick et al (1999 Nucl Med Comm. 20 pp 537-45). Both ¹³¹I and ¹²³I versions of mIBG have been used clinically, but for diagnostic imaging ¹²³I-mIBG is preferred. mIBG is an analogue of the false neurotransmitter guanethidine, which is a potent neuron-blocking agent that acts selectively on sympathetic nerves. Neuronal uptake of mIBG is predominantly via the uptake-1 mechanism at the doses typically used for imaging, with the uptake-2 mechanism becoming dominant at higher concentrations. In patients with cardiomyopathy, reduced uptake and increased washout of mIBG correlates with the degree of sympathetic dysfunction, clinical severity and prognosis. Low mIBG uptake, reduced left ventricular ejection fraction (LVEF) and circulating NE concentration are independent predictors for mortality in patients with dilated cardiomyopathy. It has been demonstrated that reduced NE reuptake plays a prominent role in the sympathetic dysfunction of advanced cardiomyopathy in comparison to less severe forms where increased release and decreased reuptake appear equally important. mIBG uptake is diminished in CHF patients as compared to controls due to altered NE uptake and storage, the uptake is very heterogenous and there is increased washout.

¹⁸F-mFBG, ¹⁸F-pFBG and ¹⁸F-mIBG are fluorinated analogues of mIBG. ¹⁸F-mFBG and ¹⁸F-pFBG can be synthesized beginning with a fluoro for nitro exchange reaction on 3- and 4-nitrobenzonitrile, respectively (Garg et al 1994 Nucl Med Biol. 21 pp 97-103). ¹⁸F-mIBG can be prepared starting from 4-cyano-2-iodo-N,N,N-trimethylanilinium trifluoromethanesulfonate by the method described by Vaidyanathan et al (1994 J Med Chem. 37 pp 3655-62). All of these ¹⁸F agents act as PET imaging agents having a similar uptake to mIBG, as described in the previous paragraph.

The synthesis of ¹¹C-CGP has been described by Brady et al (1991 Int J Rad Appl Instrum. [A]. 42 pp 621-8). This adrenergic imaging agent is a non-selective β-adrenoceptor antagonist that binds with high affinity. An example of the use of ¹¹C-CGP is in the assessment by PET imaging of in vivo changes in the number of left ventricular β-adrenergic receptor sites of patients with idiopathic cardiomyopathy. Quantitative assessment of receptor sites can also be carried out in conjunction with the use of a mathematical model (Schafers et al 1998 Eur J Nuc Med. 25 pp 435-41).

Carazolol is a high affinity β-adrenergic receptor antagonist which is relatively non-specific for the receptor subtypes. The labelling of the two enantiomers of this compound with ¹¹C, including the synthesis of the required labelling precursors, is reported by Berridge et al (1992 Int J Rad Appl Instrum B. 19 pp 563-9). Labelling of carazolol with ¹⁸F has been reported by Elsinga et al (1996 Nucl Med Biol. 23 pp 159-67). Carazolol labelled with ¹¹C or ¹⁸F can be used for β-receptor estimation with PET. The R-isomer does not accumulate in the target organs, indicating that in vivo binding of carazolol is stereoselective.

MQNB is labeled with ¹¹C by methylation of quinuclidinyl benzylate with ¹¹C-methyl iodide (Le Guludec et al 1997 Circulation 96 pp 3416-22). MQNB is a specific hydrophilic antagonist of muscarinic receptors and the ¹¹C labelled version can be used to evaluate the density and affinity constants of myocardial muscarinic receptors by PET imaging. Muscarinic receptors are part of the parsympathetic nerve system and their stimulation results in the inhibition of NE release from adrenergic neurons. Congestive heart failure is associated with upregulation of myocardial muscarinic receptors, which may be an adaption to β-agonist stimulation.

⁷⁶Br-meta-Bromobenzylguanidine (⁷⁶Br-mBBG) can be prepared from the iodinated analog (mIBG) and ⁷⁶Br—NH₄ using a Cu⁺-assisted halogen exchange reaction as reported by Loc'h et al (1994 Nucl Med Biol. 21(1) pp 49-55). ⁷⁶Br-mBBG was produced in a 60-65% radiochemical yield with a specific activity of 20 MBq/nmol. Preliminary results in rats in the same report suggest that ⁷⁶Br-mBBG can be useful for the assessment of heart catecholamine reuptake disorders with PET.

¹⁸F-FIBG was prepared by Vaidyanathan et al (1997 J Nucl Med. 38(2) pp 330-4) in four steps starting from 4-cyano-2-iodo-N,N,N-trimethylanilinium trifluoromethanesulfonate in 5% decay-corrected radiochemical yield in a total synthesis time of 130 min. The specific activity was more than 1500 Ci per mmol. In vitro binding studies showed that the percent binding of ¹⁸F-FIBG to SK—N—SH human neuroblastoma cells remained constant over a 3-log activity range and was similar to that of no carrier added ¹³¹I-mIBG. Specific and high uptake of ¹⁸F-FIBG was also seen in mouse heart and adrenals. The in vitro and in vivo properties of ¹⁸F-FIBG suggest that this compound may be a useful positron-emitting analogue of mIBG.

¹⁸F-labeled 2β-carbomethoxy-3beta-(4-chlorophenyl)-8-(-2-fluoroethyl)nortropane (¹⁸F-FECNT) is a recently developed dopamine transporter ligand with potential applications in patients with Parkinson's disease and cocaine addiction. ¹⁸F-FECNT was prepared by Deterding et al (2001 J Nucl Med. 42(2) pp 376-81) in a two-step reaction sequence. Alkylation of 1-¹⁸F-fluoro-2-tosyloxyethane with 2β-carbomethoxy-3β-(4-chlorophenyl)nortropane in dimethyl formamide at 1,350° C. for 45 min allowed ¹⁸F-FECNT, which was purified by semipreparatory, reverse phase high-performance liquid chromatography, to produce a product free from the precursor, 2β-carbomethoxy-3β-(4-chlorophenyl)nortropane and with specific activity of 56 MBq/nmol (1.5 Ci/mmol).

An “adrenergic interfering agent” as defined in the present invention is a pharmaceutical agent that interacts with a process of cardiac neurotransmission. Therefore, adrenergic interfering agents that interact with the processes relating to the synthesis, storage, release, reuptake and metabolism of NE are of particular interest in the context of the present invention. Suitable adrenergic interfering agents of the present invention include tricyclic antidepressants, β-blockers, calcium channel blockers, sympathomimetic agents and cocaine (Solanki et al Nuc Med Comm. 1992 13 pp 513-21). Preferably, the adrenergic interfering agent interacts with the same process of cardiac neurotransmission as the adrenergic imaging agent.

Tricyclic antidepressants are known to interfere with the uptake-1 mechanism, which is the main uptake mechanism for a number of adrenergic imaging agents. Examples of tricyclic antidepressants that can be used in the method of the present invention include desipramine, amitryptaline, imipramine, doxepine, loxapine, nortriptyline and trimipramine. Preferred tricyclic antidepressants of the present invention are desipramine, amitryptaline and imipramine. The β-blocker labetalol, the sympathomimetic agent ephedrine and cocaine also inhibit the uptake-1 mechanism and are therefore suitable for use in the methods of the present invention, although in reality the clinical use of cocaine in such a method may not be considered.

Various sympathomimetic agents are known to act by depleting the content of the synaptic vesicles in which NE is stored. Similarly, any adrenergic imaging agent that is known to be stored in the synaptic vesicles will also be released by the action of these agents. Examples of sympathomimetic agents that are suitable for use in the methods of the present invention include dobutamine, phenylpropranolamine, phenylephidrine and metaraminol. A preferred sympathomimetic agent of the present invention is dobutamine. The β-blocker labetalol is also known to deplete synaptic vesicle contents.

Certain calcium channel blockers have been shown to decrease the uptake of adrenergic imaging agents. Examples of calcium channel blockers that are suitable for use in the present invention include diltiazem, isradipine, nicardipine, nifedipine, nimodipine and verapimil. Preferred calcium channel blockers of the present invention are diltiazem, nifedipine and verapamil.

Administration of the adrenergic interfering agent is carried out in conjunction with obtaining one of the images of the method. The route of administration of the adrenergic interfering agent can suitably be oral or parenteral. The timing of administration may also vary and may be suitably carried out before, during or after administration of the adrenergic imaging agent. Primarily however, administration of the adrenergic interfering agent should allow it to compete with but not to block the uptake of the adrenergic imaging agent, thereby providing a “stress” on the mechanism by which the imaging agent is taken up. The effect of this stress, as reflected in the difference between the two images obtained, will be dependent on whether or not the particular aspect of cardiac neurotransmission being measured is functioning normally in the subject. Where cardiac neurotransmission is functioning normally, the uptake of adrenergic imaging agent will not be altered significantly in the stress image compared with the image obtained with adrenergic imaging agent alone (the “rest” image). Where the uptake mechanism is working at its maximal capacity in the rest image or has been rendered less efficient due to an underlying pathophysiology, reduced uptake of adrenergic imaging agent is seen in the stress image indicating a defect not visible in the rest image.

Cardioneuropathies can be broadly categorized into primary and secondary cardioneuropathies. Primary cardioneuropathies can be related to dysautonomias, heart transplantation and idiopathic ventricular tachycardia and fibrillation. Secondary cardioneuropathies can be related to dilated cardiomyopathy, coronary artery disease, hypertrophic cardiomyopathy, arrhythmogenic right ventricular cardiomyopathy, diabetes mellitus, hypertension and drug-induced cardiotoxicity. As described by Carrio (2001 J Nuc Med. 42 pp 1062-76) evaluation of the pathophysiology of all of these conditions can be done using adrenergic imaging agents. Certain patterns of uptake in rest vs. stress are reflective of particular cardiac neurotransmission status in a subject and can provide prognostic value for risk stratification relating to pump failure and/or occurrence of life-threatening arrhythmias in patients with cardioneuropathy in association with symptomatic or asymptomatic heart failure.

In a second aspect the present invention relates to a method of assessing cardiac neurotransmission in a human subject comprising;
i) administration of a non-therapeutic dose of an adrenergic interfering agent to said subject;
ii) administration to said subject of an amount suitable for in vivo imaging of an adrenergic imaging agent; and,
iii) in vivo imaging of said subject.

With this method a single image is obtained in conjunction with the administration of a non-therapeutic dose of an adrenergic interfering agent. The term “non-therapeutic dose” in the context of the present invention is taken to mean a specific dose of the adrenergic interfering agent that is low enough such that no therapeutic effect occurs, but sufficient to produce competition with the adrenergic imaging agent. This dose will depend on the particular adrenergic interfering agent used, e.g. preferred doses of the tricyclic antidepressants amitryptaline and desipramine would be between 10 and 50 mg, most preferably 25 mg. In a preferred embodiment the adrenergic interfering agent is administered as a single dose. The image produced is evaluated with respect to what would be expected from a normal subject, for instance by means of comparison with a database of normal data, such that information as to the status of cardiac neurotransmission in a subject can be derived.

Preferably the assessment of cardiac neurotransmission is used as a means to investigate the status of a cardioneuropathy in said human subject. The preferred adrenergic imaging agents and adrenergic interfering agents are as described for the first embodiment of the invention.

A third aspect of the present invention is a method for determining the viability of a region of adrenergically innervated tissue in a human subject comprising:
(i) performing in vivo imaging of said subject using an adrenergic imaging agent;
(ii) administration to said subject of an adrenergic interfering agent;
(iii) repeating step (i); and,
(iv) comparing the images obtained in steps (i) and (iii).

The adrenergically innervated tissue is preferably the myocardium and the method is preferably used to investigate the status of a cardioneuropathy in said human subject. The preferred adrenergic imaging agents and adrenergic interfering agents are as described for the first embodiment of the invention.

A fourth aspect of the present invention is a method of imaging the sympathetic innervation of a tissue of a human subject comprising:
(i) in vivo imaging with an adrenergic imaging agent;
(ii) administration of an adrenergic interfering agent;
(iii) repeating step (i); and,
(iv) comparing the images obtained in steps (i) and (iii).

The preferred tissue of this method is the myocardium and the method is preferably used to investigate the status of a cardioneuropathy in said human subject. The preferred adrenergic imaging agents and adrenergic interfering agents are as described for the first embodiment of the invention.

A fifth aspect of the present invention is a method of operating an external imaging apparatus using signal data derived from an adrenergic imaging agent previously administered to a human subject, said method being carried out both before and after the previous administration of an adrenergic interfering agent to said subject and then comparing the signal data so derived.

In the present invention the term “external imaging apparatus” is taken to mean any apparatus suitable for measuring, external to a subject, the relative distribution in said subject of an adrenergic imaging agent following its administration. Suitable external imaging apparatus of the invention include gamma cameras where the imaging moiety is a gamma emitter, PET cameras where the imaging moiety is a positron emitter and MRI scanners where the imaging moiety is a paramagnetic metal ion or a hyperpolarized NMR-active nucleus.

A sixth aspect of the present invention comprises the use of an adrenergic imaging agent in the manufacture of a medicament for use in in vivo imaging of the sympathetic innervation of a human subject wherein said in vivo imaging is carried out both before and after the administration of an adrenergic interfering agent and comparing the images so obtained.

A seventh aspect of the present invention is a kit for use in the methods of the present invention which comprises:
(i) an adrenergic interfering agent; and,
(ii) an adrenergic imaging agent in a form suitable for carrying out in vivo imaging, or a precursor thereof.

A “precursor” of an adrenergic imaging agent is a compound that can be labelled with an imaging moiety to produce an adrenergic imaging agent. When the imaging moiety comprises a non-metallic radioisotope, i.e. a gamma-emitting radioactive halogen or a positron-emitting radioactive non-metal, such a precursor suitably comprises a non-radioactive material which is designed so that chemical reaction with a convenient chemical form of the desired non-metallic radioisotope can be conducted in the minimum number of steps (ideally a single step), and without the need for significant purification (ideally no further purification) to give the desired radioactive product. Such precursors can conveniently be obtained in good chemical purity and, optionally supplied in sterile form as part of the kit of the invention.

Such kits are designed to give sterile products suitable for human administration, e.g. via direct injection into the bloodstream. Suitable kits comprise containers (e.g. septum-sealed vials) containing the adrenergic interfering agent and precursor of the adrenergic imaging agent.

The kits may optionally further comprise additional components such as radioprotectant, antimicrobial preservative, pH-adjusting agent or filler.

By the term “radioprotectant” is meant a compound which inhibits degradation reactions, such as redox processes, by trapping highly-reactive free radicals, such as oxygen-containing free radicals arising from the radiolysis of water. The radioprotectants of the present invention are suitably chosen from: ascorbic acid, para-aminobenzoic acid (i.e. 4-aminobenzoic acid), gentisic acid (i.e. 2,5-dihydroxybenzoic acid) and salts thereof with a biocompatible cation as described above.

By the term “antimicrobial preservative” is meant an agent which inhibits the growth of potentially harmful micro-organisms such as bacteria, yeasts or moulds. The antimicrobial preservative may also exhibit some bactericidal properties, depending on the dose. The main role of the antimicrobial preservative(s) of the present invention is to inhibit the growth of any such micro-organism in the pharmaceutical composition post-reconstitution, i.e. in the radioactive diagnostic product itself. The antimicrobial preservative may, however, also optionally be used to inhibit the growth of potentially harmful micro-organisms in one or more components of the kit of the present invention prior to reconstitution. Suitable antimicrobial preservatives include: the parabens, i.e. methyl, ethyl, propyl or butyl paraben or mixtures thereof; benzyl alcohol; phenol; cresol; cetrimide and thiomersal. Preferred antimicrobial preservative(s) are the parabens.

The term “pH-adjusting agent” means a compound or mixture of compounds useful to ensure that the pH of the reconstituted kit is within acceptable limits (approximately pH 4.0 to 10.5) for human administration. Suitable such pH-adjusting agents include pharmaceutically acceptable buffers, such as tricine, phosphate or TRIS [i.e. tris(hydroxymethyl)aminomethane], and pharmaceutically acceptable bases such as sodium carbonate, sodium bicarbonate or mixtures thereof. When the ligand conjugate is employed in acid salt form, the pH-adjusting agent may optionally be provided in a separate vial or container, so that the user of the kit can adjust the pH as part of a multi-step procedure.

By the term “filler” is meant a pharmaceutically acceptable bulking agent which may facilitate material handling during production and lyophilisation. Suitable fillers include inorganic salts such as sodium chloride, and water soluble sugars or sugar alcohols such as sucrose, maltose, mannitol or trehalose.

### BRIEF DESCRIPTION OF THE FIGURES

FIG. 1 illustrates the physiological route of synthesis of NE.

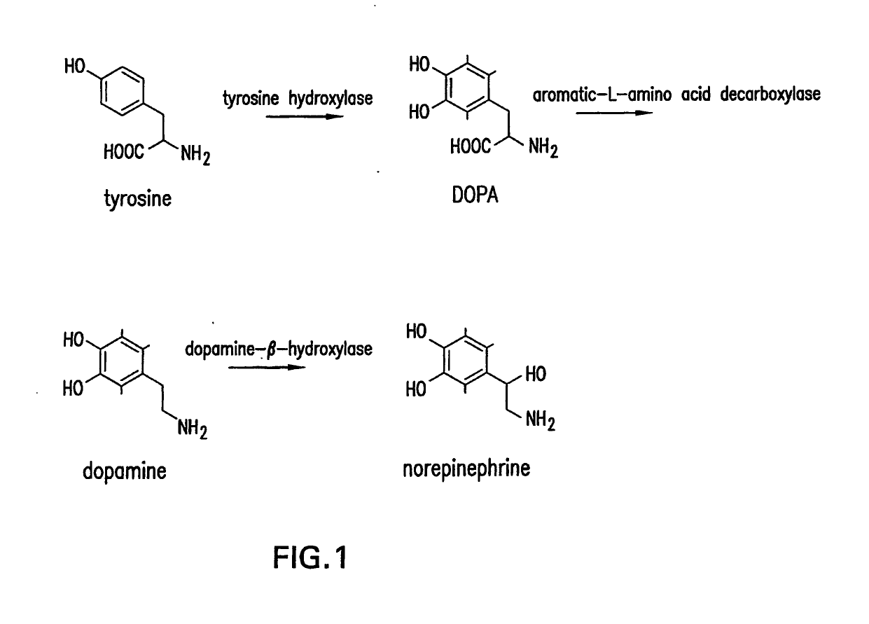

FIG. 2 shows the chemical structures of some adrenergic imaging agents of the invention.

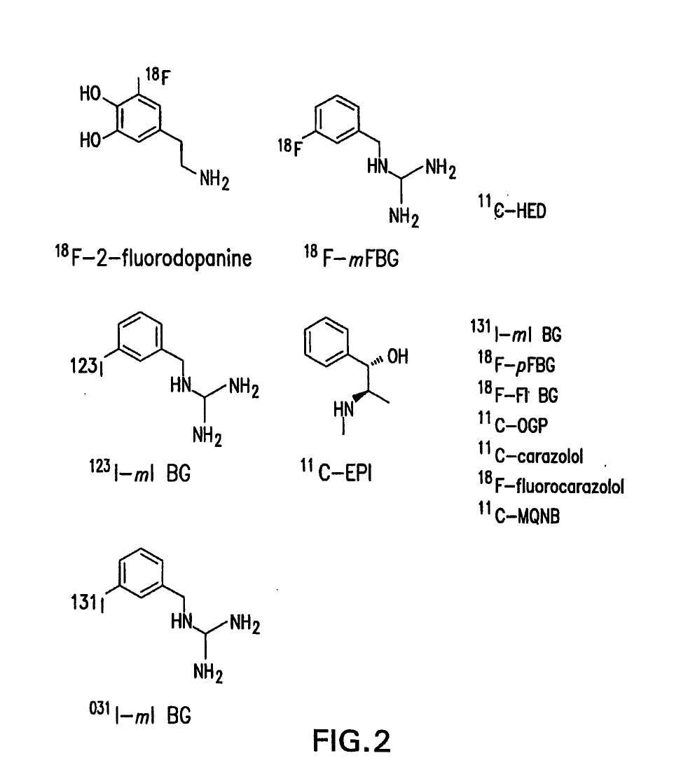

FIG. 3 illustrates ¹²³I mIBG images produced with (A) and without (B) administration of amitryptaline representative of two of the subjects studied in Example 1. When amitryptaline was administered before ¹²³I mIBG a marked decrease in the myocardial uptake of ¹²³I mIBG was seen.

FIG. 4 illustrates the ¹²³I mIBG images produced with (A) and without (B) administration of amitryptaline representative of the other two subjects studied in Example 1. There was no notable difference in the myocardial uptake of ¹²³I mIBG following administration of amitryptaline.

The difference in the response to the “stress” of amitryptaline administration between the subjects is indicative of differing degrees of cardiac neurotransmission function.

### BRIEF DESCRIPTION OF THE EXAMPLES

The invention is illustrated by the following non-limiting examples.

Example 1 describes a method of the invention in which the adrenergic imaging agent is ¹²³I mIBG and the adrenergic interfering agent is amitryptaline. Reduced uptake of ¹²³I mIBG was seen in the stress image obtained for half of the patients imaged.

It is hypothesized that reduced uptake in the stress image is as a result of partial denervation of a region of the myocardium. The mechanism for adrenergic imaging agent uptake may be working at maximal capacity for the rest image such that it becomes overwhelmed in the presence of adrenergic interfering agent resulting in significant reduction in uptake of adrenergic imaging agent. The method can therefore allow detection of milder forms of cardiac adrenergic denervation and has the potential to be a more sensitive and specific method of ¹²³I mIBG imaging. This in turn will allow better risk prognostication in terms of pump failure and likelihood of occurrence of life-threatening arrhythmias in patients with asymptomatic or symptomatic heart failure.

Example 2 describes a method of the invention in which the adrenergic imaging agent is ¹²³I mIBG and the adrenergic interfering agent is desipramine. As observed for the method of example 1, it is anticipated that this method will also provide additional diagnostic information over imaging with ¹²³I mIBG alone.

### EXAMPLES

### Example 1

### mIBG Imaging with Amitryptaline

4 patients with movement disorders and aged between 66 and 75 were selected for this study. Neurological examination raised the differential diagnosis between essential tremor and Parkinson's disease. Prior to the study, none of the patients was taking any medication known to interfere with mIBG uptake. In all patients two ¹²³I mIBG scans were performed, one of which was performed after administration of a single oral dose of 25 mg amitryptaline one hour prior to ¹²³I mIBG administration. The imaging protocol was carried out for both scans as described in the following paragraphs.

The patients were treated with 200-500 mg of potassium perchlorate 30 minutes before injection of ¹²³I mIBG. A dose of 370 MBq of ¹²³I mIBG was administered at rest through an intravenous catheter.

Anterior planar images of the thorax were obtained at 15 minutes and at 4 hours after ¹²³I mIBG injection with the subject in a supine position. The gamma camera (GE Millenium) was equipped with a low-energy, parallel-hole, general purpose collimator, and a 20% energy window on 159 KeV if ¹²³I is used.

SPECT was performed with collection of 32 projections of 30-60 seconds each, acquired over 180° orbit, with 3°-6° angle interval in a 64×64 matrix starting in the 45° right anterior oblique projection and finishing in the 45° left posterior oblique projection.

Studies were reconstructed using a Butterworth filtered backprojection technique. Three tomographic images were obtained from the SPECT study, i.e. vertical long axis slices, short axis slices, and horizontal long axis slices. Bull's eye polar map was generated from the apical to the basal short axis slices to show relative tracer distribution in the myocardium. Reconstruction was performed without attenuation and scatter correction.

The parameters used for quantification of myocardial ¹²³I-mIBG activity were heart to mediastinum ratio (HMR) and myocardial washout rate (WR). HMR is the mean pixel counts of heart region of interest (ROI) divided by the mean pixel counts of mediastinum ROI. The WR is calculated by dividing the product of myocardial counts at 4 hours minus myocardial counts at 15 minutes by myocardial counts at 15 minutes and multiplying by 100. A WR of 10% is considered normal.

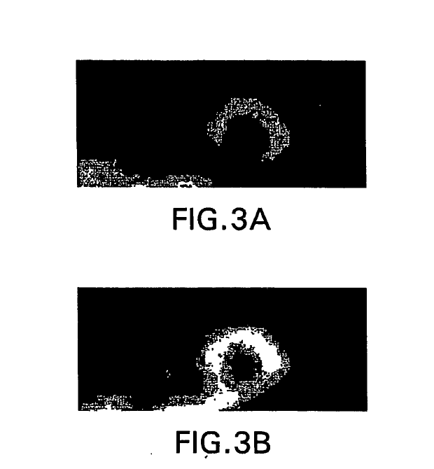

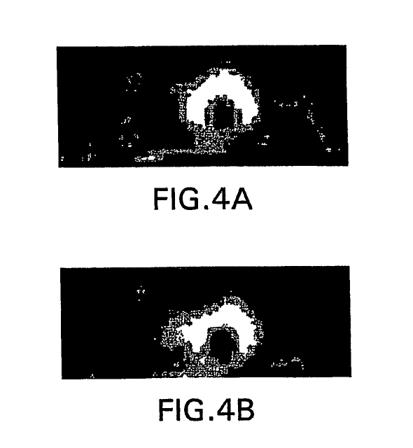

Representative images obtained in the study are illustrated in FIGS. 3 and 4.

### Example 2

### mIBG Imaging with Desipramine

20 patients of any age with a diagnosis of ischemic or non ischemic cardiomyopathy are included in the study, matched by an equal number of asymptomatic age matched controls. Patients with ischemic cardiomyopathy have already been intervened for maximal possible augmentation of myocardial perfusion via coronary artery bypass grafting, or angioplasty. All subjects continue to receive standard and maximal medical care for heart failure and other co-morbidities from their respective primary care physicians.

Prior to the start of the study all medications are reviewed and potential drug interactions with desipramine and ¹²³I mIBG uptake identified. Drugs which can confound the interpretation and can be stopped without adversely altering the clinical profile of the patient are withheld. However if such a step is not possible (digoxin, labetalol, ACE inhibitors) the results are interpreted keeping in view the medications being administered. The study comprises a 2-day imaging protocol.

Desipramine hydrochloride is administered via an intravenous infusion to the patients and normal controls. The cumulative dosage of desipramine administered is 0.25-0.5 mg/kg and the infusion lasts for 15-20 minutes.

Thirty minutes after desipramine administration, 370 MBq of ¹²³I mIBG is administered to each patient after the desipramine infusion and images are obtained at 15-30 minutes and at 4 hours after ¹²³I mIBG administration. As in Example 1, the WR is also calculated.

24 hours later, 370 MBq of ¹²³I mIBG is administered again and same set of images is repeated.

What is claimed is:

1. A method of assessing cardiac neurotransmission in a human subject comprising:
i) administration to said subject of an amount suitable for in vivo imaging of an adrenergic imaging agent;
ii) in vivo imaging of said subject using said adrenergic imaging agent;
iii) administration of an adrenergic interfering agent to said subject;
iv) repeating steps (i) and (ii); and,
v) comparing the images obtained in steps (ii) and (iv).

2. The method of claim 1 wherein said cardiac neurotransmission is assessed to investigate the status of a cardioneuropathy in said human subject.

3. The method of claim 2 wherein said cardioneuropathy is a primary cardioneuropathy related to:
(i) a dysautonomia;
(ii) heart transplantation; or,
(iii) idiopathic ventricular tachycardia and fibrillation.

4. The method of claim 2 wherein said cardioneuropathy is a secondary cardioneuropathy related to:
(i) dilated cardiomyopathy;
(ii) coronary artery disease;
(iii) hypertrophic cardiomyopathy;
(iv) arrhythmogenic right ventricular cardiomyopathy;
(v) diabetes mellitus;
(vi) hypertension; or,
(vii) drug-induced cadriotoxicity.

5. The method of claim 1 wherein said adrenergic interfering agent is selected from:
(i) tricyclic antidepressants;
(ii) beta blockers;
(iii) calcium channel blockers;
(iv) sympathomimetic agents; and,
(v) cocaine.

6. The method of claim 5 wherein said adrenergic interfering agent is a tricyclic antidepressant selected from desipramine, amitryptaline and imipramine.

7. The method of claim 6 wherein said adrenergic interfering agent is amitryptaline.

8. The method of claim 1 wherein said adrenergic imaging agent is selected from labelled forms of mIBG, mFBG, hydroxyephedrine, ephedrine, fluorodopamine, CGP, carazolol and MQNB.

9. The method of claim 8 wherein said adrenergic imaging agent is radioiodinated mIBG.

10. The method of claim 9 wherein said adrenergic imaging agent is ¹²³I mIBG.

11. The method of claim 1 wherein said in vivo imaging is external imaging carried out by SPECT or PET.

12. The method of claim 11 wherein said external imaging is carried out by SPECT.

13. A method of assessing cardiac neurotransmission in a human subject comprising:
i) administration of a non-therapeutic dose of an adrenergic interfering agent to said subject;
ii) administration to said subject of an amount suitable for in vivo imaging of an adrenergic imaging agent; and,
iii) in vivo imaging of said subject.

14. The method of claim 13 wherein said cardiac neurotransmission is assessed to investigate the status of a cardioneuropathy in said human subject.

15. The method of claim 14 wherein said cardioneuropathy is a primary cardioneuropathy related to:
(i) a dysautonomia;
(ii) heart transplantation; or,
(iii) idiopathic ventricular tachycardia and fibrillation.

16. The method of claim 14 wherein said cardioneuropathy is a secondary cardioneuropathy related to:
(i) dilated cardiomyopathy;
(ii) coronary artery disease;
(iii) hypertrophic cardiomyopathy;
(iv) arrhythmogenic right ventricular cardiomyopathy;
(v) diabetes mellitus;
(vi) hypertension; or,
(vii) drug-induced cadriotoxicity.

17. The method of claim 13 wherein said adrenergic interfering agent is selected from:
(i) tricyclic antidepressants;
(ii) beta blockers;
(iii) calcium channel blockers;
(iv) sympathomimetic agents; and,
(v) cocaine.

18. The method if claim 17 wherein said adrenergic interfering agent is a tricyclic antidepressant selected from desipramine, amitryptaline and imipramine.

19. The method of claim 18 wherein said adrenergic interfering agent is amitryptaline and the non-therapeutic dose is between 10 and 50 mg.

20. The method of claim 13 wherein said adrenergic imaging agent is selected from labelled forms of mIBG, mFBG, hydroxyephedrine, ephedrine, fluorodopamine, CGP, carazolol and MQNB.

21. The method of claim 20 wherein said adrenergic imaging agent is radioiodinated mIBG.

22. The method of claim 21 wherein said adrenergic imaging agent is ¹²³I mIBG.

23. The method of claim 13 wherein said in vivo imaging is external imaging carried out by SPECT or PET.

24. The method of claim 23 wherein said external imaging is carried out by SPECT.

25-37. (canceled)

38. A method of imaging the sympathetic innervation of a tissue of a human subject comprising:
(i) in vivo imaging with an adrenergic imaging agent;
(ii) administration of an adrenergic interfering agent;
(iii) repeating step (i); and,
(iv) comparing the images obtained in steps (i) and (iii).

39. The method of claim 38 wherein said tissue is the myocardium.

40. The method of claim 38 wherein said sympathetic innervation is imaged to investigate the status of a cardioneuropathy in said human subject.

41. The method of claim 40 wherein said cardioneuropathy is a primary cardioneuropathy related to:
(i) a dysautonomia;
(ii) heart transplantation; or,
(iii) idiopathic ventricular tachycardia and fibrillation.

42. The method of claim 40 wherein said cardioneuropathy is a secondary cardioneuropathy related to:
(i) dilated cardiomyopathy;
(ii) coronary artery disease;
(iii) hypertrophic cardiomyopathy;
(iv) arrhythmogenic right ventricular cardiomyopathy;
(v) diabetes mellitus;
(vi) hypertension; or,
(vii) drug-induced cadriotoxicity.

43. The method of claim 38 wherein said adrenergic interfering agent is selected from:
(i) tricyclic antidepressants;
(ii) beta blockers;
(iii) calcium channel blockers;
(iv) sympathomimetic agents; and,
(v) cocaine.

44. The method of claim 43 wherein said adrenergic interfering agent is a tricyclic antidepressant selected from desipramine, amitryptaline and imipramine.

45. The method of claim 44 wherein said adrenergic interfering agent is amitryptaline.

46. The method of claim 45 wherein said adrenergic imaging agent is selected from labelled forms of mIBG, mFBG, hydroxyephedrine, ephedrine, fluorodopamine, CGP, carazolol and MQNB.

47. The method of claim 38 wherein said adrenergic imaging agent is radioiodinated mIBG.

48. The method of claim 47 wherein said adrenergic imaging agent is ¹²³I mIBG.

49. The method of claim 38 wherein said in vivo imaging is external imaging carried out by SPECT or PET.

50. The method of claim 49 wherein said external imaging is carried out by SPECT.

51-72. (canceled)

73. A kit for use in the method of claim 1 which comprises:
(i) an adrenergic interfering agent; and,
(ii) an adrenergic imaging agent in a form suitable for carrying out said in vivo imaging steps, or a precursor thereof.

74. The kit of claim 73 wherein said adrenergic interfering agent is selected from:
(i) tricyclic antidepressants;
(ii) beta blockers;
(iii) calcium channel blockers;
(iv) sympathomimetic agents; and,
(v) cocaine.

75. The kit of claim 74 wherein said adrenergic interfering agent is a tricyclic antidepressant selected from desipramine, amitryptaline and imipramine.

76. The kit of claim 75 wherein said adrenergic interfering agent is amitryptaline.

77. The kit of claim 73 wherein said adrenergic imaging agent is selected from labelled forms of mIBG, mFBG, hydroxyephedrine, ephedrine, fluorodopamine, CGP, carazolol and MQNB.

78. The kit of claim 77 wherein said adrenergic imaging agent is radioiodinated mIBG.

79. The kit of claim 78 wherein said adrenergic imaging agent is ¹²³I mIBG.
